(19) United States
(12) Patent Application Publication
(10) Pub. No.: US 2006/0189683 A1
(43) Pub. Date: Aug. 24, 2006

(54) Parenteral formulation of mycophenolic acid, a salt or prodrug thereof

(76) Inventor: Markus Ahlheim, Staufen (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/548,737

(22) PCT Filed: Mar. 31, 2004
(86) PCT No.: PCT/EP04/03423

§ 371 Date: Sep. 9, 2005

(30) Foreign Application Priority Data
Apr. 1, 2003 (GB) 0307553.8

(51) Int. Cl. A61K 31/365

(52) U.S. Cl. 514/469

(57) ABSTRACT

A pharmaceutical composition in the form of powder or a lyophilized composition for injection suitable for parenteral administration comprising MPA, a salt or a produg thereof.

The present invention relates to novel pharmaceutical compositions suitable for parenteral administration comprising mycophenolic acid, a salt or a prodrug thereof.

Mycophenolic acid, also referred to herein as MPA, is a natural product of complex structure and particular sensitivity, which has anti-tumor, anti-viral, immunosuppressive, anti-psoriatic, anti-inflammatory, and anti-cancer activity.

High molecular weight derivatives such as the morpholinomethylester of MPA, also known as mycophenolate mofetil, have been made in order to increase bioavailability. Mycophenolate mofetil is commercially used as an immunosuppressant for the treatment or prevention of organ or tissue transplant rejection.

WO 97/38689 describes a pharmaceutical composition, e.g. capsules, comprising a mycophenolate salt. The composition is adapted to release the mycophenolate salt in the upper part of the intestinal tract.

An enteric-coated tablet of mycophenolate sodium is known under the tradename Myfortic®.

For acute situations or in case an oral administration of MPA, a salt or a prodrug thereof is not possible, e.g. prior to or immediately after surgery, a pharmaceutical composition suitable for parenteral administration, e.g. suitable for intravenous, subcutaneous or intramuscular administration, is desired.

Applicants have found that at physiological pH, e.g. at a pH of about 6.8 to about 8.0, pharmaceutical compositions comprising MPA, a salt or a prodrug thereof in solution are not sufficiently stable upon storage, e.g. for about 2 weeks at about 25° C. or above, or after heat treatment, e.g. for 15 min at about 121° C.

A pharmaceutical composition in the form of a powder comprising MPA, a salt or a prodrug thereof is very stable, e.g. for about 30 months at about 25° C. or below, and may easily be dissolved with a suitable solvent, preferentially with water for injection, to reconstitute a solution suitable for parenteral administration.

Accordingly, the present invention provides a pharmaceutical composition in the form of powder or a lyophilized composition for parenteral administration comprising MPA, a salt or a prodrug thereof. By “for parenteral administration” it is meant that the composition is suitable for parenteral administration e.g. after reconstitution as a solution in a physiologically acceptable solvent. Preferably the composition comprises MPA or a mycophenolate salt.

Preferably the composition is for injection. Thus the invention also provides a pharmaceutical composition in form of a powder for injection and a solution for parenteral administration, e.g. for injection, obtainable by reconstitution of said composition in a suitable solvent.

According to a preferred embodiment of the invention, there is provided a pharmaceutical composition in the form of a powder, e.g. suitable for injection, comprising

a) MPA, a salt or a prodrug thereof,

b) a pharmaceutically acceptable buffer,

c) a lyophilisation bulking agent, and

d) a pharmaceutically acceptable basic compound.

Preferably the composition consists essentially of the above components.

According to an alternative embodiment of the invention, there is provided a pharmaceutical solution for parenteral administration comprising components (a), (b), (c) and (d) and (e) a physiologically acceptable solvent.

A suitable mycophenolate salt may be e.g. cationic salts of MPA, e.g. alkali metal salts, especially the sodium salt, alkaline earth metal salts, an ammonium salt or a salt with an organic base may be used. According to the present invention, preferably the mono-sodium salt may be used.

Both prior to and after lyophilisation, the MPA, salt or prodrug thereof, e.g. the mono-sodium salt may be in crystalline or amorphous form. For example the MPA or mycophenolate salt may be in any one of the crystalline forms disclosed in PCT/EP04/00354. The mono-sodium salt may be obtained in crystalline form by recrystallization, e.g. from acetone/ethanol if necessary with water; m.p. 189-191° C.

By “pharmaceutically acceptable buffer” is meant a compound which resists a change in pH when H⁺ or OH⁻ is added. A buffering agent can be a single compound or a combination of compounds. Examples of a pharmaceutically acceptable buffer are e.g. a compound which allows to buffer the solution for parenteral administration to a pH of 6.8 to 8.0, for example sodium phosphate, potassium phosphate, disodium hydrogenphosphate, dipotassium hydrogen phosphate, sodium dihydrogen phosphate, potassium dihydrogen phosphate, or phosphoric acid.

By “lyophilisation bulking agent” is meant a compound which acts as bulk, provides a matrix structure and/or stabilizes the agent agent (e.g. by slowing or preventing decomposition of the active agent) during and/or after lyophilisation. Suitable lyophilisation bulking agents include e.g. mannitol, saccharose, lactose, fructose, glucose, trehalose, dextrans, phospholipids, lecithins, gelatine, amino acids such as glycine or cellulose.

The basic compound is preferably selected in such a way that the solution for parenteral administration is adjusted to a pH of 6.8 to 8.0. Preferably the basic compound is a base, e.g. sodium hydroxide or potassium hydroxide, or a basic salt e.g. sodium hydrogen carbonate, sodium carbonate, potassium hydrogen carbonate, or potassium carbonate.

The solvent (e) may be water for injection, physiological saline or an aqueous saline of 5% glucose. By water for injection is meant clear, colorless, and odorless water containing no added substances and purified by reverse osmosis or distillation (see Physician's Desk Reference).

The amount of MPA, a salt or a prodrug thereof in the powder for injection of the invention is from about 0.1 mg to about 100 mg, preferably from about 30 mg to about 60 mg, based on a total volume of 1 ml of injectable solution. The upper limit of concentration of MPA, a salt or a prodrug thereof in the solution for injection depends upon the solubility of the drug in the solvent. Preferably, no solubilizing aid, is present.

The amount of lyophilisation bulking agent in the solution of the invention is from about 5 to about 100 mg/ml. Preferably, the lyophilisation bulking agent is present in an amount that after reconstitution in solvent (e) an isotonic solution for injection is obtained.

The choice of buffer and the amount of buffer and base depend upon the desired pH of the solution for injection. Preferably, the pH of the solution of the invention is adjusted to be within the range of from about 6.8 to about 8.0, most preferably about 7.5.

The compositions of the invention may contain additional excipients commonly employed in parenteral compositions in order to provide the required stability and therapeutic efficacy. Excipients may include e.g. antioxidants.

Antioxidants may be employed to protect the active agent from oxidative degradation particularly under the accelerated conditions of thermal sterilisation. Antoxidants may be selected from any of those compounds known in the art. Similarly, the amount of antioxidant employed can be determined using routine experimentation. Preferably, the compositions of the invention do not contain an antioxidant.

Reference is made to the extensive literature on the subject for these and other excipients and procedures mentioned herein, see in particular Handbook of Pharmaceutical Excipients, Second Edition, edited by Ainley Wade and Paul J. Weller, American Pharmaceutical Association, Washington, USA and Pharmaceutical Press, London; and Lexikon der Hilfsstoffe für Pharmazie, Kosmetik and angrenzende Gebiete edited by H. P. Fiedler, 4th Edition, Editio Cantor, Aulendorf and earlier editions which are incorporated herein by reference.

Preferably, the composition of the invention contains as active ingredient only MPA, a salt or a prodrug thereof.

Procedures which may be used to prepare the compositions of the invention may be conventional or known in the art or based on such procedures e.g. those described in L. Lachman et al. The Theory and Practice of Industrial Pharmacy, 3rd Ed, 1986, H. Sucker et al, Pharmazeutische Technologie, Thieme, 1991, Hager's Handbuch der pharmazeutischen Praxis, 4th Ed. (Springer Verlag, 1971) and Remington's Pharmaceutical Sciences, 13th Ed., (Mack Publ., Co., 1970) or later editions.

Typically, the MPA, a salt or a prodrug thereof, the buffer (b) and the lyophilization bulking agent (c) are dissolved in an aqueous solvent, preferentially in water for injection, and the pH is adjusted with the base (d). The resulting solution may then be diluted with water to make it up to the final desired volume. The resulting solution may be filtered through a sterile filter, e.g. a modified polyvinylidene fluoride membrane, e.g. Durapore®, and charged in vials, e.g. glass vials. The solution is freeze-dried by a conventional method under aseptic conditions. The resulting powder for injection may be used to reconstitute the desired solution for parenteral administration shortly before administration: the powder is mixed with the desired amount of solvent (e) e.g. with water for injection, prior to administration.

Preferably, during above preparation oxygen (air) is displaced from contact with the solution of MPA, a salt or a prodrug thereof. This is usually carried out by purging with, e.g. nitrogen, a container holding the solution.

The invention also provides an injection kit comprising a lyophilized preparation, e.g. as disclosed herein, and a physiologically acceptable solvent.

The compositions of the invention are useful as immunosuppressants as indicated by standard tests.

The activity and characteristics of the compositions of the invention may be indicated in standard
a) clinical trials, e.g. observing the first acute rejection episodes or treatment failure six months after transplant of kidneys or maintaining a rejection-free state within 6 months after initiation of treatment with the invention. The compositions of the invention are administered at a dose in the range of 0.05 to 3 g/day, preferably 0.2 to 3 g/day, more preferably 0.5 to 2 g/day e.g. about 1.5 g/day and decrease the acute rejection rates when administered during the period around transplant surgery, and maintain a rejection-free state in patients who are 3 months or more after transplantation. Thus the compositions of the invention may be administered during the initial 72 hours after transplantation at dose of about 0.5 g administered twice a day in combination with a conventional steroid and cyclosporin, e.g. as NEORALR for which the cyclosporin dose is the conventional dose e.g. ca 8±3 mg/kg for renal transplants. The steroid dose is to be administered at about 2.5 mg/kg for 4 days after transplant, 1 mg/kg thereafter for 1 week, 0.6 mg/kg thereafter for 2 weeks thereafter 0.3 mg/kg for 1 month for prednisone, and in
b) animal trials e.g. observing the kidney allograft reaction in rat. In this test one kidney from a female fisher 344 rat is transplanted onto the renal vessel of a unilaterally (left side) nephrectomized WF recipient rat using an end-to-end anastomosis. Ureteric ananstomosis is also end-to-end. Treatment commences on the day of transplantation and is continued for 14 days. A contralateral nephrectomy is done seven days after transplantation, leaving the recipient relying on the performance of the donor kidney. Survival of the graft recipient is taken as the parameter for a functional graft. Typical doses of the compositions of the invention are from about 1 to 30 mg/kg.

The compositions of the invention are particularly useful for the following conditions:
a) Treatment or prevention of organ, tissue or cellular allograft or xenograft transplant rejection, e.g. for the treatment of recipients of e.g. heart, lung, combined heart-lung, liver, kidney, bowel, pancreatic, skin, pancreatic islet cell, neural cell or corneal transplant; including treatment or prevention of acute rejection; treatment and prevention of hyperacute rejection, e.g. as associated with xenograft rejection; and treatment or prevention of chronic rejection, e.g. as associated with graft-vessel disease, or restenosis. The compositions of the invention are also indicated for the treatment or prevention of graft-versus-host disease, such as following bone marrow transplantation.
b) Treatment or prevention of autoimmune diseases, e.g. immune-mediated diseases and inflammatory conditions, in particular inflammatory conditions with an etiology including an immunological component such as arthritis (for example rheumatoid arthritis, arthritis chronica progrediente and arthritis deformans) and rheumatic diseases. Specific immune-mediated disease for which the compositions of the invention may be employed include, autoimmune hematological disorders, including, but not limited to hemolytic anaemia, aplastic anaemia, pure red cell anaemia and idiopathic thrombocytopenia), systemic lupus erythematosus, polychondritis, sclerodoma, Wegener granulosis, dermatomyositis, polymyositis, chronic active hepatitis, primary bilary cirrhosis, myasthenia gravis, psoriasis, Steven-Johnson syndrome, pemphigus, idiophatic sprue, inflammatory bowel disease (including e.g. ulcerative colitis and Crohn's disease), endocrine ophthalmophathy, Graves disease, sarcoidosis, multiple sclerosis, juvenile diabetes (diabetes mellitus type 1), non-infectious uveitis (anterior and posterior), keratoconjunctivitis sicca and vernal keratoconjunctivitis, interstitial lung fibrosis, psoriatic arthritis, vasculitis, glomerulonephritides (with and without nephrotic syndrome, e.g. including idiophatic nephrotic syndrome or minimal change nephropathy) and juvenile dermatomyositis.

Appropriate dosage of the composition of the invention will of course vary, e.g. depending on the condition to be treated (for example the disease type or the nature of resistance), the drug used, the effect desired and the mode of administration.

When given continuously, an effective amount of drug may be given in two or three doses spread over time such as by parenteral administration, e.g. intravenous drip or intramuscular or subcutaneous injection(s) with the total daily dose being spread across the portion or the entire administration period. When given by subcutaneous injection, it is most preferably administered from 3 times per week up to 3 times a day, preferably twice a week up to once or twice daily.

The composition of the invention preferably is suitable for intravenous administration. The immediate response of this form of administration is highly desirable in acute situations. Furthermore, as no absorption process is involved, the dose or blood concentration of active agent may be obtained with greater accuracy and speed.

In general, satisfactory results are obtained on administration, e.g. intravenous administration, at dosages of the order of from about 1 to about 30 mg mycophenolate salt per kg animal body weight per day, administered once or in divided doses up to 4 times per day.

Suitable daily dosages for patients are thus in the order of 0.05 to 3 g/day, preferably 0.2 to 3 g/day, more preferably 0.5 to 2 g/day e.g. about 1.5 g/day mycophenolate salt.

In another aspect, the present invention provides an injection kit comprising a composition of the invention in form of a powder for injection and a suitable solvent.

The compositions of the invention comprising a therapeutically effective amount of MPA, a salt or a prodrug thereof may be administered as the sole active ingredient or with another immunosuppressant e.g. together with simultaneous or separate administration of other immunosuppressants, e.g. in immunosuppressive applications such as prevention or treatment of graft vs. host disease, transplant rejection, or immune-mediated diseases. For example, the compositions of the invention may be used in combination with a cyclosporin or an ascomycin, or their immunosuppressive analogs or derivatives, e.g. cyclosporin A, Is a Tx247, FK-506 (tacrolimus), etc., a mTOR inhibitor, e.g. rapamycin or a derivative thereof, e.g. 40-O-(2-hydroxyethyl)-rapamycin, a derivative as disclosed e.g. in WO 95/14023 and 99/15530, e.g. ABT578, or rapalogs as disclosed e.g. in WO 98/02441 and WO 01/14387, e.g. AP23573, AP23464, AP23675, AP23841 or TAFA-93; a S1P receptor agonist having accelerating lymphocyte homing properties, e.g. FTY720 (2-amino-2-[2-(4-octylphenyl) ethyl]propane-1,3-diol in free form or in a pharmaceutically salt form, e.g. the hydrochloride) or an analogue thereof; corticosteroids; cyclophosphamide; azathioprine; methotrexate; brequinar; leflunomide; mizoribine; deoxyspergualin; or immunosuppressive monoclonal antibodies, e.g., monoclonal antibodies to leukocyte receptors, e.g. MHC, CD2, CD3, CD4, CD7, CD25, CD28, CTLA4, B7, CD40, CD45, or CD58 or to their ligands; or other immunomodulatory compounds, e.g. CTLA4-1 g, or a mutant thereof e.g. LEA29Y. A preferred combination comprises a composition of the invention and rapamycin or a derivative thereof, e.g. as indicated above, e.g. 40-O-(2-hydroxyethyl)-rapamycin, and/or a S1P receptor agonist having accelerating lymphocyte homing properties, e.g. FTY720.

Accordingly in a further aspect the present invention provides a method of immuno-suppressing a subject which comprises administering a composition according to the invention, e.g. an intravenous composition, to a subject in need of such immunosuppression, optionally with the simultaneous, sequential or separate administration of another immuno-suppressant or immunomodulatory compound, e.g. as disclosed above.

When the compositions of the invention are co-administered with such other immuno-suppressants the dosages of the other immunosuppressants may be reduced e.g. to one-half to one-third their dosages when used alone.

Representative doses for cyclosporin A to be used are e.g. 1 to 10 mg/kg/day, e.g. 1 to 2 mg/kg/day. Representative doses for 40-O-(2-hydroxyethyl)-rapamycin are e.g. 0.75 to 5 mg bid. Representative doses for (2-amino-2-[2-(4-octylphenyl) ethyl]propane-1,3-diol hydrochloride are e.g. 1.25 to 10 mg per day.

The following Examples serve to illustrate the invention.

### EXAMPLE 1

Disodium hydrogenphosphate (8.70 mg) and mannitol (125.1 mg) are dissolved in water for injection (about 1.5 ml), while the solution is purged with nitrogen. Then mycophenolate sodium (160.35 mg) is added, the solution is adjusted with sodium hydroxide to pH 7.5 and water for injection up to 3.0 ml is added. Under aseptic conditions, the solution is filtered through a Durapore® sterile filter with a pore size≦0.22 μm and filled into vials. The solution is freeze-dried under aseptic conditions to give a powder for injection.

### EXAMPLE 2

The powder for injection of example 1 is used to reconstitute a solution for injection with 5 ml of water for injection.

The solution is clear, exhibits a pH of 7.5 and is suitable for intravenous, subcutaneous and intramuscular administration.

### EXAMPLE 3

12 ml of the solution of example 2 is given to 12 stable renal transplant patients as an intravenous continuous infusion into an arm vein over 30 min with a constant infusion rate of 0.4 ml/min.

Blood samples are taken for 36 h after dosing at the following time points: 0, 10 min, 20 min, 30 min, 45 min, 1.0 h, 1.5 h, 2.0 h, 4.0 h, 8.0 h, 12.0 h, 24.0 h, 36.0 h from the onset of infusion.

The medication is well tolerated in these renal transplant patients.

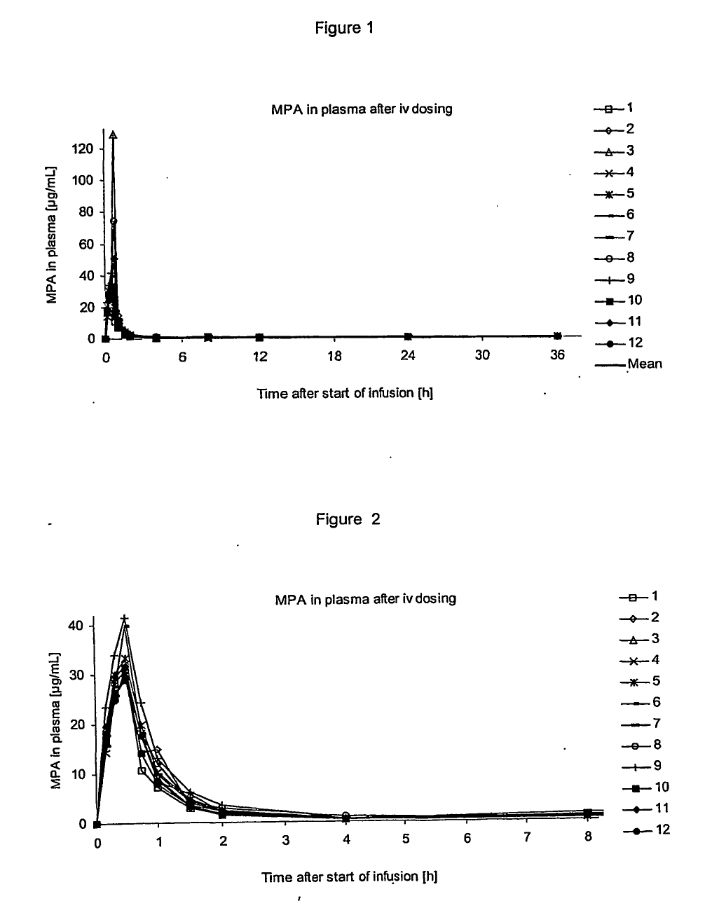

Plasma levels of MPA are given in FIGS. 1 and 2.

Mean MPA AUC₀₋ₜ is 42.1 μg h/ml and interpatient variability for AUC₀₋ₜ is less than 25%. Mean t_{1/2} is 9.68 h.

In further examples, the procedure of examples 1 to 3 is repeated but mycophenolate sodium is replaced by mycophenolate mofetil.

1. A pharmaceutical composition in the form of powder or a lyophilized composition for parenteral administration comprising MPA, a salt or a prodrug thereof.

2. A composition according to claim 1 comprising
a) MPA, a salt or a prodrug thereof,
b) a pharmaceutically acceptable buffer,
c) a lyophilisation bulking agent, and
d) a pharmaceutically acceptable base.

3. A composition according to claim 2 wherein the lyophilisation bulking agent is mannitol.

4. A composition according to any preceding claim wherein the mycophenolate salt is mycophenolate sodium.

5. A composition according to any preceding claim which forms a solution of pH 6.8 to 8.0 when reconstituted in water.

6. A solution for parenteral administration obtainable by reconstitution of a composition according to any preceding claim in a physiologically acceptable solvent.

7. A solution according to claim 6 wherein the MPA, a salt or a prodrug thereof is in a concentration of about 0.1 to about 100 mg/ml.

8. A solution according to claim 7 wherein MPA, a salt or a prodrug thereof is in a concentration of about 30 mg/ml.

9. An injection kit comprising a composition according to any one of claims 1 to 5 and a physiologically acceptable solvent.

10. Use of a pharmaceutical composition according to any one of claims 1 to 5 for the preparation of a medicament for immunosuppression, particularly for prevention or treatment of native or transgenic organ, tissue or cellular allograft transplant rejection, for the treatment or prevention of immune-mediated and/or inflammatory disease, optionally with the simultaneous, sequential or separate administration of another immuno-suppressant.

11. A method of immunosuppressing a subject in need of immunosuppression which comprises administering a solution according to any one of claims 6 to 8 to the subject, optionally with the simultaneous, sequential or separate administration of another immunosuppressant.
